(19) United States
(12) Patent Application Publication
(10) Pub. No.: US 2026/0234424 A1
(43) Pub. Date: Aug. 13, 2026

(54) Self-Activating Dipping Powder

(71) Applicants: Thong Huu Vu, Vista, CA (US); Hung Viet Hoang, Lake Mary, FL (US); Thanh Hung Tran, Lake Mary, FL (US); Phong Tran Deo, Orlando, FL (US)

(72) Inventors: Thong Huu Vu, Vista, CA (US); Hung Viet Hoang, Lake Mary, FL (US); Thanh Hung Tran, Lake Mary, FL (US); Phong Tran Deo, Orlando, FL (US)

(21) Appl. No.: 19/287,805

(22) Filed: Jul. 31, 2025

Related U.S. Application Data
(60) Provisional application No. 63/677,949, filed on Jul. 31, 2024.

(51) Int. Cl. C09D 7/61; A61K 8/02; A61K 8/25; A61K 8/81; A61Q 3/02; C09D 7/40; C09D 7/63; C09D 133/12

(52) U.S. Cl. CPC C09D 7/61; A61K 8/022; A61K 8/25; A61K 8/8152; A61Q 3/02; C09D 7/63; C09D 7/69; C09D 7/70; C09D 133/12

(57) ABSTRACT

A self-activating dipping powder for artificial nail applications is disclosed. The powder comprises glass beads having a pH greater than 7 and less than 10, preferably soda lime borosilicate. The glass beads, which may be solid or hollow with mean particle sizes of 20-55 microns, provide basic surface sites that initiate polymerization of acid-stabilized cyanoacrylate dip bases without the need for a separate activator. Incorporation of the glass beads shortens dry times from over one hour to under ten minutes, allowing activator-free dipping applications. In sculpting applications, the inclusion of glass beads increases the working time window, produces lighter nails, and improves filing and removability. The invention further provides 2-in-1 acrylic and dipping powders, enabling both sculpting and dipping procedures using a single formulation while improving application efficiency and wearer comfort.

### FIELD OF THE INVENTION

This invention arises in the field of formulating acrylic powders in cosmetic nail industry. Acrylic powders have been used at least in two different cosmetic nail applications: sculpting nail and dipping nail, each of which requires a corresponding liquid part, sculpting liquid and dip liquid respectively. Both sculpting nail and dipping nail are considered artificial nails.

### BACKGROUND OF THE INVENTION

Acrylic powders contain polymer powders. The polymer powders are made of a homopolymer(s), a copolymer(s), or a mixture of homopolymers and/or copolymers in the family of polyalkyl (meth)acrylates. The “akyl” can be methyl, ethyl, propyl, butyl, etc . . . . The “(meth)acrylates” can be acrylate or methacrylate. Most common polymers used in acrylic powders are polymethyl methacrylate (PMMA), polyethyl methacrylate (PEMA), and their copolymers in various ratios. Particle sizes of the polymers come with a mixture of certain size distribution. Mean particle sizes of acrylic powders are normally in the range of about 40 to 60 microns. Polymer powders have a good flow property such that they can be poured. In another word, they flow almost like a fluid. The fluid-like flow the powder indicating that the powder particles are in geometrical shapes that have low friction such as rounded, or spherical.

Acrylic powder also contains a catalyst (initiator). Benzoyl peroxide (herein “BPO”) is the most used catalyst in acrylic powders. It is common that polymer powders supplied to the formulators already have a certain amount of BPO in them. BPO may exist inside the powder particle (internal) and/or in between the particles (external.) The more BPO in the powder, the faster the speed of polymerization reaction in sculpting system. In dipping system, the role of BPO is not as critical. Beside polyalkyl (meth)acrylate polymer as primary polymer, acrylic powders sometimes contain non-acrylic polymer (such as polyvinyl acetate) as secondary polymer. Acrylic powders may also contain cosmetic pigments of various kinds for purposes such as opacity, color, and visual effects.

Acrylic powders are formulated to have desired properties. Properties of acrylic powder before, during, and after mixed with liquids are considered. The powder should have proper particle size, stable color, easy to work with, and safe to be used. The final nail set should have acceptable appearances, color stability, wearability, and easy removal.

Nail appearances may include surface smoothness, opacity and color. Choice of pigments in the powder may include organic, inorganic, lake pigments, and/or effect pigments. Color of acrylic powders may fade and/or turn to yellow tone. The fading and/or yellowing of acrylic powder may arise due to oxidation of pigments in the present of benzoyl peroxide. Fading and yellowing may also occur right after acrylic powder is mixed with its liquid part and/or during the time of wearing the nail.

Wearability herein is the ability of an artificial nail to last during an expected wear time. An artificial nail is expected to last without breaking, chipping, cracking, or lifting for at least two weeks. Wearability also includes the comfortability felt by the wearer.

Workability herein is the ability to be worked with. The time windows for sculpting or dipping activities to be completed vary according to the formulae of the powder and of the liquid. During pick-up step, the speed of partial dissolution of the powder particles with the liquid is critical. During sculpting step, the speed of hardening the bead, which relates to the speed of polymerization reactions, is critical. The speed of polymerization reaction varies not only by compositions of the acrylic powder and the corresponding liquid part, but also by the ambient conditions in term of temperature, humidity, and oxygen level. The hotter the ambient temperature the faster the polymerization reaction is. As with dipping, the higher moisture content in the air helps cyanoacrylate polymerized faster. Ambient oxygen is known to inhibit polymerization on the surface of sculpting nail. Working time window should be not too short because it gives higher chance for failure. Another aspect of workability is the ease-to-file which relates to the hardness of the artificial nail.

Removability herein is the ability of removing artificial nail out from the natural nail. The removal can be done either mechanically or chemically. It is common that artificial nail is removed partially by mechanical mean first, then by chemical mean. Removability is directly related to the physical hardness and the strength of chemical bonds established in the artificial nail. It is a result of the chemical make-up of acrylic powder and sculpting nail liquid, the mix-ratio, and the environmental exposures during wear time.

Basic procedure of sculpting an acrylic nail involves picking up, transferring, sculpting, filing, and smoothing.

The powder-liquid mixture picked up from the brush forms a heterogeneous mixture, called a “bead.” The size and wetness of the bead should be controlled for best results. The amount of powder in the bead should be in proportion (mix ratio) to the amount of liquid. The size, wetness, and texture of the bead are all visible to applicator's eyes. They are key factors that contribute to the success of subsequent steps and the durability of the finished acrylic nail set.

During the pick-up step, the liquid held on the brush hairs contacts and absorbs the powder forming a pasty bead that clings on the brush. The pastiness comes from the process of powder particles being dissolved partially by monomer in sculpting liquid. That makes the viscosity of the mixture get higher. The set of rheological behaviors of the bead, contributes to the ease of the sculpt step. The degree of runniness, stickiness, gumminess of the bead plays roles in ability to sculpt (sculpt-ability). Rheology of the bead change rapidly over time due to the polymerization activated by interaction of BPO in the powder and DMPT (or the like) in sculpting liquid.

The bead is then transferred onto the nail. The success of the transferring requires cohesiveness of materials within the bead to be stronger than the adhesion between the bead and the surface of brush hair. It is then sculpted using sculpting brush. The sculpting actions involve padding, moving different portions of the bead to different areas of the nail. Thickness and length of the artificial nail is roughly defined during this step. Depending on the nail length the “apex” thickness and location should be appropriately chosen. Sculpting would be easy if the bead is malleable (not too runny, not too soft, not too hard.) Due to on-going polymerization process, the bead gets thicker (in viscosity) thus becomes less malleable over time. The window of time during which the bead is sculp-table can be relatively too short or too long depending on the speed of polymerization reaction and the working speed of the applicator. A high-skill applicator would be able to finish the sculpting step successfully within a short time while a lower-skill may miss the window.

When the sculpted nail gets harden enough, filing can be done using hand-file or electrical-file. If the bead is still gummy, the file would be clogged up with gummy material. If the bead is too hard, the force applied on it must be stronger and the file material must be harder and sharper. Easy-to-file is therefore a needed property of sculpting and dipping systems. This step sometimes can be skipped if the sculpting-by-brush step was done well enough. Lastly, smoothing by buffing might be done.

The sculpting procedure with associated requirements in each step listed above indicates that (a) physical properties of acrylic power and monomer liquid, (b) chemical make-up of acrylic power and monomer liquid, (c) tools, (d) environmental factor, particularly temperature and oxygen concentration in the air, and (e) nail technician's actions are critical factors that contribute to the workability, durability, and removability of an acrylic nail.

Wearability is how well the nail set lasts on the wearer until the next the salon visit (2-4 weeks.) Most of the time, the wearer would request a “fill” (or professionally called “rebalance”) in which a newer layer of sculpting nail overlay on top of the previous one and restructuring the “apex.” Sometimes, the wearer may need a “full set”, the service in which the old acrylic nail would be removed completely, and a new set of acrylic nails is applied. Due to important factors such as time, money, and nail health, full sets are often avoided unless necessary. After many times of “fill”, there co-exist layers of old and new acrylic on a nail. The oldest layer could be as old as 3-6 months. Appropriate products (powder, liquid), applications, and wearer's physical and chemical exposures would allow the feasibility of having multiple “fills.” After the artificial nail is completed, the nails expose to environmental conditions such as temperature, humidity, sunlight, household chemicals. They all play rolls in the degradations of acrylic nail. For example, the nails may become harder, more brittle, yellower, faded. That may come from the degradation of the polymer. It may also come from activities of the trace of unreacted BPO, DMPT, monomers, and their by-products.

The existence and popularity of dipping nail occurred decades after sculpting nail. Conventional dip system consists of 4 different liquids contained in different bottles. The main ones are “Dip base” (often called bottle #2) which contains mainly alkyl cyanoacrylate (such as ethyl cyanoacrylate) and “Activator” (often called bottle #3) which contains an amine-type activating ingredient (such as DMPT) and organic solvent (such as ethyl acetate.)

During dip nail process, dip base is applied on designated nail area on the nail. The nail is then dipped into the powder. Excess powder not sticking to dip base is flicked off or brush off. Each time of doing so, a layer of pasty, heterogenous mixture of cyanoacrylate and powder is formed. One or two more layers on top of the first layer can be applied. A skilled applicator can create the shape of nail using multiple layers, starting with small area first at about the point of “apex”. Subsequence layers are applied with wider areas. The final area would cover the whole nail. As the result, the nail thickness at “apex” is the highest. The layers connect to adjacent one almost uniformly, meaning it is difficult to observe the border between them. This is due to partial solubility of the powder when it contacts with dip base.

Conventionally, following dipping step is the application of activator, without it the dip nail would not “dry” or “harden” within an allowable time frame of less than 20 minutes (a salon visit is normally about 1 hour). The hardening of dip nail is the result of polymerization process of ethyl cyanoacrylate. It has 3 major steps—initiation, propagation, and termination which are expressed in simple expressions, without the intention of being academically correct, as shown in Reaction #1, #2, and #3 below.

Ethyl cyanoacrylate (CH₂═C(CN)(COOEt)) in the dip base reacts to water (moisture) in ambient air. Hydroxide ions (OH⁻), generated from the autoionization of water, initiates the reaction by forming a carbanion as shown in Reaction #1.

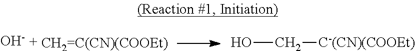

The availability of OH⁻ influences the speed of reaction #1. Reaction #1 is expected to occur fast in high level of moisture in the air or with alkaline media, slowly in neutral, and inhibited in acidic media. Dip base in a bottle will be used for many dip applications with the bottle opened and the brush going in and out so many times. Conventional dip base comes with a little acidic activity which stabilizes the cyanoacrylate maintains usefulness of dip base's bottle for multiple usages. Dip base with acidic activity is called acid-stabilized dip base.

The carbanion formed in initiation attacks additional ECA monomers, forming a growing polymer chain as shown in Reaction #2.

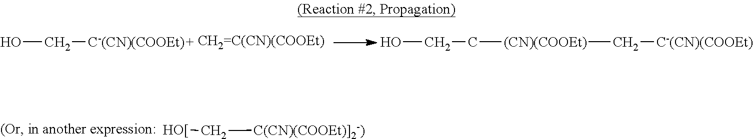

The propagation keeps on continue linking more cyanoacrylate monomers to the polymer chain until it is terminated (by either protonation from water, or chain transfer, or the exhaustion of available monomer) as shown in Reaction #3.

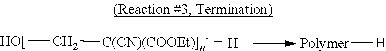

Dry time, the time it takes for the dip nail to dry or harden, is therefore depending on the polymerization reaction rate in which temperature play a role. Temperature would impact the dry time basing on Arrhenius behavior. For simple estimation of reaction speeds, “Q10 rule” has been widely adopted in industrial applications. Q10 rule says for each 10° C. increase approximately doubles the reaction rate given appropriate and narrow range of temperatures.

Due to the nature of serving people, normal nail salons are operated in temperature range of that is rather comfortable such as 20-30° C. Sometimes, some salons may operate in wider range (saving energy cost) such as 15° C.-35° C., in such case the “extreme” temperature is 10° C. lower or higher than 25° C. So, a dry time at about 25° C. can be used to estimate its value at 15° C. and 35° C. by multiple it by 2 or divide it by 2, respectively. The condition in our experiments is 30-60% humidity and 22-28° C.

Without activator or other alkaline agent, dry time of dip nail using acid-stabilized dip base is more than 1 hour. With activator, it is about 5-15 minutes, well within the allowable time window. This is because the initiation step is now much faster. DMPT does a much better job than OH⁻ as a nucleophile as shown in Reaction #4. Hence Reaction #4 is faster than Reaction #1. The lone pair on the nitrogen atom of DMPT (CH₃-C₆H₄—N(CH₃)₂) acts as a Lewis base, attacking the β-carbon of ethyl cyanoacrylate. This generates a carbanion stabilized by electron-withdrawing CN and COOR groups.

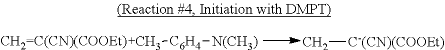

Speed of reaction should be controlled properly. Too-fast-too-soon of reactions would result in too short of cyanoacrylate polymer's chain length leading to fragile, easy-cracking nails. It also may cause heat shock on the nail due to too much heat (from polymerization reaction which is exothermic) releasing out in too short period of time. Too-slow-too-late of reactions would result in too big of dry time, too soft and too weak of the nails. Speed of reaction can be controlled by concentration of DMPT in activator. Physical properties of dip nail depend on the make-ups of dipping powder, dip base, and activator.

Initially dipping nail systems utilize acrylic sculpting powders without alternation. Later, improvements were made on acrylic powders to make them more dipping friendly. For example, the particle size of acrylic powder was a little too big for dipping. Customized acrylic powders specialized for dipping had smaller particle size than that of regular acrylic powder. That customized dipping powder coexisted with conventional acrylic powder. Later, for economic reasons (such as reduction of powder inventories in salons, nail supplies, and manufacturers) there came 2-in-1 acrylic powders, which can be used for both sculpting and dipping. And that is how most acrylic powders are advertised in the market at the time of this application.

### Issues and Difficulties:

Activator in dip systems have unpleasant odor and may cause eye irritation. Activator in dipping application add more money, time, discomfort, and health risks to applicators and wearers. There existed a need of eliminating the activator in dip system. Filing or removing sculpting nails (and dipping nails to less extent) cost time and efforts of the nail applicator and causes discomfort to the wearers. There is a need of having acrylic powder that is filled more easily. Because of small working window due to speed of reaction, sculpting nail job requires high skill and experienced. There is a need of having sculpting powder that has slower speed of reaction to give the workers a little longer working time. The peroxide in acrylic powder does not help polymerization in dipping powder but causes yellowing and fading issues. There is a need of having dipping powder that has as less BPO or even no BPO.

### The Inventions:

In one aspect of our invention, self-activated dipping powders are achieved using glass beads. Dip nails using acid-stabilized dip base and dipping powders containing glass beads can be dried and harden a time frame that is small enough to be appropriate for dipping procedure.

The glass beads in our experiments are either solid or hollow, with spherical shapes, and mean particle sizes ranging within 25 to 60 microns. Sometimes, particle sizes are expressed in term of D10, D50, and D90. In the experiments, mean particle size or D50 will be displayed depending on their availabilities. The glass beads have pH number obtained from manufacture or collected from measurements using pH paper and solutions of 5 wt % of glass beads in water. The glass materials are disclosed as soda lime borosilicate glass.

It is known to the skilled in the art that acid-stabilized cyanoacrylates polymerize strongly in the presence of moisture and basic media such as glass beads with pH greater than 7 as shown below. Both the OH-ion (which would be more available in basic media) and the basic surface of the glass beads can act as nucleophile as shown in Reaction #5. Such mechanism polymerization of dip base without the need of activator containing DMPT.

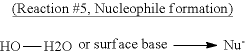

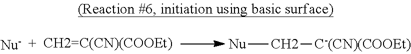

Formula for common soda lime borosilicate glass can be represented as SiO₂·B₂O₃·Na₂O. The exact composition varies based on the specific formulation, but typically it consists of: SiO₂ the main constituent (typically 60-80%), B₂O₃ 5-15%, Na₂O 5-10%, CaO 5-10%. Some soda lime borosilicate glass may also contain Al₂O₃. The present of Na₂O in soda lime borosilicate formula may explain why the glass beads have alkaline activity.

### Tests and Examples

Here are the materials used in the experiments, their sources, their compositions (if available.) Acrylic & Dipping Clear Powder (P7) from Keystone Cosmetics and Sciences, NJ, USA. PMMA, PEMA, BPO, Silica. Dip base from Chisel Nail Art®, FL, USA. Ethyl Cyanoacrylate, PMMA. Activator from Chisel Nail Art®, FL, USA. Ethyl Acetate, DMPT. SG3000 and SG4000 are Spheriglass® A-Glass solid glass spheres from Potters® Industries LLC, PA, USA. S32HS, K37, K15 are Glass Bubbles (hollow) from 3M™ Advanced Material. St. Paul, Minnesota, USA. Baking Soda from local supermarket.

Properties of glass beads are listed in Table #1. Numbers related to particle size (mean particle size, D50) are from the manufactures. A pH test method is adopted from 3M's literature which measure pH of “solution” of 5 weight percentage of the solid in DI water. However, instead of using pH meter which is not available, pH paper is used. Water is tested alone giving pH 7. pH of 7-8 means pH that is greater than 7 but smaller than 8. pH 9-10 means pH greater than 9 and smaller than 10.

TABLE #1
Materials having pH greater than 7 and smaller than 10 used in experiments.

| | | | | | | Baking |
|---|---|---|---|---|---|---|
| Product Name | SG4000 | SG3000 | S32HS | K37 | K15 | Soda |
| pH (paper, 5% in | 9 | 7-8 | 9-10 | 9 | 9-10 | 9 |
| DI Water) | | | | | | |
| Manufacture's | N/A | N/A | 9.1-9.9 | 9.1-9.9 | 9.1-9.9 | N/A |
| specification: pH | | | | | | |
| D50 (microns) | 33 | 19 | 25 | 45 | 60 | N/A |
| Mean particle size | 37 | 27 | N/A | N/A | N/A | N/A |
| (microns) | | | | | | |
| True density (g/cc) | N/A | N/A | 0.32 | 0.37 | 0.15 | N/A |

In the experiments, a test method for dry time was adopted. Timer starts right after three layers of dip coating is formed on a plastic nail tip. Unless specified, no accelerator is applied. A toothpick is used for poking and scratching the coating with high force from the hand. The time when the coating no longer be poked through nor scratched is recorded as “Dry Time.” In each test, glass beads were added in P7 at various weight percentages followed by 1 minute of mixing. Dry Times were measured in seconds then converted to minutes. Data of different glass beads are shown in Table #2, #3, #4, #5 and #6. Data for baking soda shown in Table #7, which can be used as a contrast data. In those 6 tables, equations and R squares of best-fit trend line are shown. The obtained equations can be used to predict Dry Time for untested scenarios.

TABLE #2

| | Dry Time | y = 18.335x^{-2.006} |
|---|---|---|
| SG4000 (%) | (minutes) | R² = 0.9933 |
| 1 | 22 | 18 |
| 2 | 3.8 | 4.6 |
| 4 | 1.5 | 1.1 |
| 6 | 0.30 | 0.50 |
| 8 | 0.25 | 0.28 |
| 12 | 0.18 | 0.13 |

TABLE #3

| | Dry Time | y = 280.35x^{-1.978} |
|---|---|---|
| SG3000 (%) | (minutes) | R² = 0.995 |
| 4 | 18 | 18.1 |
| 8 | 5.55 | 4.6 |
| 12 | 1.58 | 2.1 |
| 20 | 0.77 | 0.7 |
| 40 | 0.20 | 0.2 |

TABLE #4

| | Dry Time | y = 24.1x−^{1.52} |
|---|---|---|
| S32HS (%) | (minutes) | R² = 0.9989 |
| 1 | 25 | 24.1 |
| 2 | 8 | 8.4 |
| 4 | 2.8 | 2.9 |
| 5 | 2.3 | 2.1 |

TABLE #5

| | Dry Time | y = 7.591x^{-0.703} |
|---|---|---|
| K37 (%) | (minutes) | R² = 0.9879 |
| 5 | 2.5 | 2.4 |
| 9 | 1.5 | 1.6 |
| 13 | 1.25 | 1.3 |
| 17 | 1.08 | 1.0 |
| 20 | 1 | 0.9 |
| 23 | 0.83 | 0.8 |
| 29 | 0.67 | 0.7 |
| 100 | 0.25 | 0.3 |

TABLE #6

| | Dry Time | y = 29.553x^{-0.879} |
|---|---|---|
| K15 (%) | (min) | R² = 0.9328 |
| 100 | 0.58 | 0.5 |
| 50 | 1.00 | 0.9 |
| 33 | 1.33 | 1.4 |
| 25 | 1.42 | 1.7 |
| 20 | 1.67 | 2.1 |
| 14.3 | 3.0 | 2.9 |
| 10 | 5.0 | 3.9 |

TABLE #7

| Baking Soda | Dry Time | y = 688.72x^{-2.414} |
|---|---|---|
| (%) | (min) | R² = 0.991 |
| 5 | 17 | 14.1 |
| 7.5 | 5 | 5.3 |
| 10 | 2 | 2.7 |
| 20 | 0.6 | 0.5 |

Dry Time of P7 by itself is found beyond 60 minutes. Dry Time of P7 using Activator is about 10 minutes. Dry Times of glass beads by themselves (100 weight %) are equal or smaller than 1 minutes. The summary of Dry Time with glass beads' weight percentages (0, 1, 5, 10, 20, 100.) is shown in Table #8.

TABLE #8
Summary of Dry Times (rounded minutes)

| | | | | | | Baking |
|---|---|---|---|---|---|---|
| Glass % | SG4000 | SG3000 | S32HS | K37 | K15 | Soda |
| 0 | >60 | >60 | >60 | >60 | >60 | >60 |
| 1 | 22 | 280 | 25 | 8 | 46 | 689 |
| 5 | 1 | 12 | 2 | 3 | 9 | 17 |
| 10 | 0 | 3 | 1 | 1 | 5 | 2 |
| 20 | 0 | 1 | 0 | 1 | 2 | 1 |
| 100 | 0 | 0 | 0 | 0 | 1 | 0 |

Predictions of weight % to achieve Dry Times of equal or less than milestone time windows (20 minutes, 10 minutes, 5 minutes) are shown in Table #9.

TABLE #9
Prediction of Glass Beads content (wt
%) to achieve milestones of Dry Times

| | | | | | | Baking |
|---|---|---|---|---|---|---|
| Prediction | SG4000 | SG3000 | S32HS | K37 | K15 | Soda |
| Dry Time <=20 | 1% | 4% | 1.5% | 0.50% | 3% | 4.5% |
| minutes (%) | | | | | | |
| Dry Time <=10 | 1.5% | 6% | 2% | 1% | 5% | 6% |
| minutes (%) | | | | | | |
| Dry Time <=5 | 2% | 9% | 3% | 2% | 10% | 8% |
| minutes (%) | | | | | | |

Table #9 together Table #1 tells us that, in general, the more pH of the solid glass beads, the smaller the Dry Time. Within the group of hollow glass beads having the same pH of 9-10, S32HS has smaller particle size and Dry Time compared with K15. Interesting observation found in which baking soda does not show advantage above SG3000 which has lower pH. Unlike baking soda which is totally soluble in water, the lower measured pH of the glass is due to its insolubility in water. The present of glass beads, with weight percentage sometimes as small as 1% can bring Dry Time to 10 minus or less. In conclusion, the present of the glass beads makes the regular dipping powder such as P7 becomes a self-activating dipping powder.

### Prior Arts:

Montgomery's “Method of making artificial nails,” (U.S. Pat. No. 5,098,690) mentioned about glass powder in an acrylic powder. The nature, amount, and purpose of the glass were not specified.

There exists special dip base that help eliminate the need of activator. “Super Dip Base” of Chisel Nail Art® is an example. Using Super Dip Base, the Dry Time conventional acrylic powder is within 20 minutes. Its pH is 7 (measured by pH paper with 5 weight % mixed in water.) So, “Super Dip Base” is not an acid-stabilized dip base. Therefore, it has problem with easily get polymerized after multiple uses in high humidity environment. It solved the problem from the dip base's side. This invention solves the problem from the powder's side.

### INVENTIONS

In one aspect of our invention, self-activating dipping powders are achieved by adding glass beads with pH greater than 7 to dipping powders that need activator otherwise.

In another aspect of our invention, self-activating dipping powders are formulated using glass bead, acrylic polymer as secondary, and a non-acrylic primary polymer such as CAP 482-05 (cellulose acetate propionate) and CAB 551-0.01 (cellulose acetate butyrate). In Example #1, Example #2, and Example #3, CAP and CAB do not have BPO and are products of Eastman.

### Example #1

| | |
|---|---|
| CAP | 60 wt % |
| P7 | 35 wt % |
| SG4000 | 5 wt % |

### Example #2

| | |
|---|---|
| CAB | 50 wt % |
| P7 | 35 wt % |
| SG4000 | 15 wt % |

### Example #3

| | |
|---|---|
| CAP | 100 wt % |
| SG4000 | 6 wt % |

Dry Time is 4 minutes for Example #1, 2 minutes for Example #2, 8 minutes for Example #3. All three examples give Dry Times that are less than the typical (about) 10 minutes of conventional Dry Time with Activator.

Neither CAP or CAB in the experiments has BPO. Example #3 shows BPO playing no important role in Dry Time. Cutting down BPO content would lessen the problem of fading and other unwanted affects due to its present in the powder.

The particles of powders in Examples #1, #2, and especially Example #3 are seen too big and not rounded. The dip nails look rough, not smooth due to CAP or CAB's the particle shapes (not round) and sizes (bigger than 55 microns.) Particle shape of the powders should be round (preferably spherical) and size should be about 55 microns or less for non-acrylic powder to work well in dip system.

Another aspect of the invention is increasing workability of acrylic sculpting powder by adding glass beads to the powder. With some amount of glass beads in an acrylic powder the workable window of time for pick-up and sculpting steps are larger. The larger window of time helps applicators have enough time to work successfully on the pick-up and the sculpting steps. The resulting sculpted nails are also easier to be filed and removed, especially when hollow glass beads (glass bubbles) are used such as in Example #4.

### Example #4

| | |
|---|---|
| P7 | 80 wt % |
| K37 (glass bubble of size 45 microns) | 20 wt %. |

A simple test method for relative cure time for sculpting system is created. Acrylic powder samples with different weight percentage of 45-micron glass bubbles are mixed with sculpting liquid in corresponding mix ratio such that all test samples would have similar viscosity after mixing with sculpting liquid. Start the stopwatch right after mixing. Detect the exotherm heat released by touching the bottom of the weighing boat which contains the mixture. Record the time at which the earliest warmness detected. Use a toothpick to poke through the pasty mixture. Record the time at which the toothpick cannot poke through. While doing the routine poking, detect and record the time when the bottom of the weighing boat feels the hottest. Times are recorded in seconds, then converted to minutes. The times of feeling of the heat, the time of feeling the hardness in this test show relative cure times which can be monitored in the simplest way. The goal of the test is finding correlation between glass bubble contents and cure time.

It is found that sculpting powders with more glass bubble content have smaller mix ratios and longer cure times. The relative cure times from this test indicate the trends of actual curing progression during sculpting procedure. Test data and trends are shown in Table #10 (which include data for Example #4) is shown below. It is observed that the more glass bubble weight percentage the longer it takes for the exotherm indicators to take place, hence the longer it takes for polymerization reactions to occur and complete.

TABLE #10
Correlation of glass bubble contents with mix ratio and the
key timeline of exothermic polymerization in sculpting nail.

| Glass Bubble | | Warm | Hot | Harden |
|---|---|---|---|---|
| (Wt %) | Mix Ratio | (min) | (min) | (min) |
| 0 | 1.34 | 3.1 | 3.5 | 4.1 |
| 5 | 1.31 | 3.2 | 3.8 | 4.1 |
| 9 | 1.28 | 3.2 | 3.9 | 4.3 |
| 13 | 1.25 | 3.5 | 4.1 | 4.6 |
| 17 | 1.21 | 4.0 | 4.5 | 4.7 |
| 20 | 1.16 | 4.3 | 4.8 | 4.8 |
| 23 | 1.13 | 4.7 | 5.2 | 5.3 |
| 29 | 1.05 | 4.8 | 5.5 | 5.8 |
| Trend found: | Decreasing | Increasing | Increasing | Increasing |

Wearers of sculpting nail with hollow glass bubbles in the powder (as shown in Example #4) feel lighter, not as tight, closer to “natural feel” than conventional sculpting nail wearers. Sculpting nails using Example #4 powder are also found to be softer, filed more easily, and removed more easily both mechanically and chemically.

In another aspect of the invention, acrylic powder with glass beads can be formulated to be used for both sculpting and dipping applications. Optimization on glass beads' types (either solid or hollow), pH, weight percentages, glass sizes against key properties and performances of sculpting and dipping nail would give optimized formulae for 2-in-1 powder. For lightness, it is better to use hollow glass beads (glass bubbles) as opposed to solid glass beads. For smoothness, it is better to use smaller mean particle size glass beads. Glass bead's size would be best to be equal or smaller than acrylic powder's size (mean particle of around 50 microns.) Very small particle should be use with health and safety in mind. Regarding wearability, it was found that compositions with 40 (as in Example #5) or more weight percentage of glass beads would produce sculpting nails that are fragile. They crack and break more easily than the ones having less glass beads.

### Example #5

| | |
|---|---|
| P7 | 60 wt % |
| Glass Bubble K37 | 40 wt % |

### Example #6

| | |
|---|---|
| P7 | 90 wt % |
| Solid glass SG4000 | 2 wt % |
| Solid glass SG3000 | 3 wt % |

Color of artificial nail also needs to be considered. Even though P7 gives clear sculpting and dipping nails, both Example #4 and Example #5 give sculpting and dipping nails opaque and white shades, which due to the incorporation of glass bubbles. Dip nail made from Example #6 looks as transparent as dip nail from P7. So, glass bubbles should be used only in powders aiming for opaque and/or white shades.

1: Composition of self-activating dipping powder comprising glass beads that have pH greater than 7 and smaller than 10.

2: Composition of self-activating dipping powder of claim 1 wherein the glass beads are of sodium lime borosilicate type.

3: Composition of self-activating dipping powder of claim 2 wherein glass beads are in spherical shapes.

4: Composition of self-activating dipping powder of claim 3 wherein spherical glass beads having mean particle size from 20 to 55 microns.

5: Composition of self-activating dipping powder of claim 4 wherein spherical glass beads having mean particle size from 25 to 45 microns.

6: Composition of self-activating dipping powder of claim 5 wherein glass beads' content is in the range of 0.5 to 35 weight percentage.

7: Composition of self-activating dipping powder of claim 6 wherein glass beads' content is in the range of 1 to 25 weight percentage.

8: Composition of self-activating dipping powder of claim 4 wherein the polymer has non-acrylic polymer(s) as primary polymer.

9: Composition of self-activating dipping powder of claim 8 containing less than 1 weight percent of benzoyl peroxide.

10: Composition of 2-in-1 acrylic and dipping powder comprising glass beads that have pH greater than 7 and smaller than 10.

11: Composition of 2-in-1 acrylic and dipping powder of claim 10 wherein the glass beads are of sodium lime borosilicate type.

12: Composition of 2-in-1 acrylic and dipping powder of claim 11 wherein the glass beads are in spherical shapes.

13: Composition of 2-in-1 acrylic and dipping powder of claim 12 wherein the glass beads having mean particle size in the range of 20-55 microns.

14: Composition of 2-in-1 acrylic and dipping powder of claim 13 wherein glass beads' content is in the range of 0.5 to 35 weight percentage.
